(19) United States
(12) Patent Application Publication
(10) Pub. No.: US 2021/0012864 A1
(43) Pub. Date: Jan. 14, 2021

(54) PREDICTING PARTICIPANT DROP-OUT AND COMPLIANCE SCORES FOR CLINICAL TRIALS

(71) Applicant: International Business Machines Corporation, Armonk, NY (US)

(72) Inventors: Celia Cintas, Nairobi (KE); William Ogallo, Nairobi (KE); Aisha Walcott, Nairobi (KE); Sekou Lionel Remy, Nairobi (KE)

(21) Appl. No.: 16/510,340

(22) Filed: Jul. 12, 2019

(51) Int. Cl. G16H 10/20; G16H 20/10; G16H 50/20; G06F 17/27; G10L 25/90; G06K 9/00

(52) U.S. Cl. CPC G16H 10/20; G16H 20/10; G06K 9/00221; G06F 17/2785; G10L 25/90; G16H 50/20

(57) ABSTRACT

An apparatus obtains participant sentiment data including at least one of: a text conversation between a participant and an investigator in a clinical trial; a video conversation between the participant and the investigator; and the participant's social network information; extracts and normalizes a sentiment score from the participant sentiment data; generates a compliance score for the participant by using a trained regressor on at least the sentiment score; compares the compliance score to a lower threshold and to a higher threshold; selects an action from a decision tree in response to the compliance score; and facilitates the action.

### BACKGROUND

The present invention relates to the electrical, electronic, and computer arts, and more specifically, to use of artificial intelligence to automate management of healthcare clinical trials.

Every year, medical device manufacturers and pharmaceutical manufacturers undertake clinical trials to establish safety and efficacy of their products. Clinical trials are required by government regulators in major markets. The estimated annual cost of clinical trials, in the United States alone, is on the order of $50 billion. Clinical trials take six to nine years on average to complete, and may account for almost 40% of total therapy costs.

The value of a clinical trial for establishing product safety and efficacy depends on the extent to which trial participants comply with the clinical trial protocol.

### SUMMARY

Principles of the invention provide techniques for predicting participant drop-out and compliance scores for clinical trials. In one aspect, an exemplary method includes obtaining participant sentiment data including at least one of: a text conversation between a participant and an investigator in a clinical trial; a video conversation between the participant and the investigator; and the participant's social network information; extracting and normalizing a sentiment score from the participant sentiment data; generating a compliance score for the participant by using a trained regressor on at least the sentiment score; comparing the compliance score to a lower threshold and to a higher threshold; and selecting an action from a decision tree. The decision tree includes: in case the compliance score is less than the lower threshold, updating a principal investigator's dashboard with the compliance score; in case the compliance score is greater than or equal to the lower threshold and less than or equal to the higher threshold, updating the principal investigator's dashboard with a deviation alert and the compliance score; and in case the compliance score exceeds the higher threshold, sending a termination alert to the principal investigator's dashboard and terminating the participant's involvement in the clinical trial. A further step includes facilitating the action.

One or more embodiments of the invention or elements thereof can be implemented in the form of a computer program product including a computer readable storage medium with computer usable program code for performing or otherwise facilitating the method steps indicated. Furthermore, one or more embodiments of the invention or elements thereof can be implemented in the form of a system (or apparatus) including a memory that embodies computer executable instructions, and at least one processor that is coupled to the memory and operative by the instructions to perform or to otherwise facilitate exemplary method steps. Yet further, in another aspect, one or more embodiments of the invention or elements thereof can be implemented in the form of means for carrying out one or more of the method steps described herein; the means can include (i) hardware module(s), (ii) software module(s) stored in a tangible computer readable storage medium (or multiple such media) and implemented on a hardware processor, or (iii) a combination of (i) and (ii); any of (i)-(iii) implement the specific techniques set forth herein.

As used herein, “facilitating” an action includes performing the action, making the action easier, helping to carry the action out, or causing the action to be performed. Thus, by way of example and not limitation, instructions executing on one processor might facilitate an action carried out by instructions executing on a remote processor, by sending appropriate data or commands to cause or aid the action to be performed. For the avoidance of doubt, where an actor facilitates an action by other than performing the action, the action is nevertheless performed by some entity or combination of entities.

In view of the foregoing, techniques of the present invention can provide substantial beneficial technical effects. For example, one or more embodiments provide one or more of:

Enhanced prediction of clinical trial participant compliance with clinical trial protocols.

Enhanced prediction of clinical trial participant dropout from the clinical trial.

Early institution of participant interventions to mitigate risks of clinical trial protocol non-compliance or dropout.

Early determination of clinical analysis eligibility based on severity of protocol non-compliance.

The method can be used in site-less and hybrid clinical trial environments.

Enhanced prediction of compliance score based on social network interactions, posts.

In site-less and hybrid environments the dropout score is based on sentiment analysis over video, audio and text conversations.

These and other features and advantages of the present invention will become apparent from the following detailed description of illustrative embodiments thereof, which is to be read in connection with the accompanying drawings.

### BRIEF DESCRIPTION OF THE DRAWINGS

FIG. 1 depicts in a flowchart steps of a method for predicting participant drop-out and compliance scores for a clinical trial, according to an exemplary embodiment;

FIG. 2 depicts schematically a system for implementing the method of FIG. 1, according to an exemplary embodiment;

FIG. 3 depicts formulas for calculating a participant's dropout score and compliance score;

FIG. 4 depicts schematically an example of operation of the method and system of FIGS. 1 and 2;

FIG. 5 depicts in a flowchart another example of how an embodiment of the invention operates; and

FIG. 6 depicts a computer system that may be useful in implementing one or more aspects and/or elements of the invention.

### DETAILED DESCRIPTION

Patient dropout rates in pharmaceutical clinical trials are estimated to range between 15 to 40 percent of enrolled participants, depending on the trial phase. Retention of patients throughout the entire cycle of a clinical trial is crucial for scientific and economic reasons. Poor retention negatively impacts the overall amount of valuable data for regulatory submissions and increases costs for pharmaceutical companies. Non-adherence, protocol deviations, and protocol violations are costly to the pharmaceutical industry as future clients demand health outcomes research that demonstrates a product's benefit, safety, and other properties.

It would be helpful to be able to predict during a clinical trial which participants will adhere to the clinical trial protocol (good compliance) and which participants will drop out of the trial or otherwise fail to adhere to the trial protocol (non-compliance). Interventions could then be implemented to improve retention and compliance of the participants predicted to be non-compliant.

Accordingly, embodiments of the invention generate prediction scores for participants' dropout risk (“dropout score”) and likelihood of protocol non-compliance (“compliance score”) based on sentiment analysis of video, audio and text conversations, user interaction logs, and accomplished checkpoints during the current time at the clinical trial.

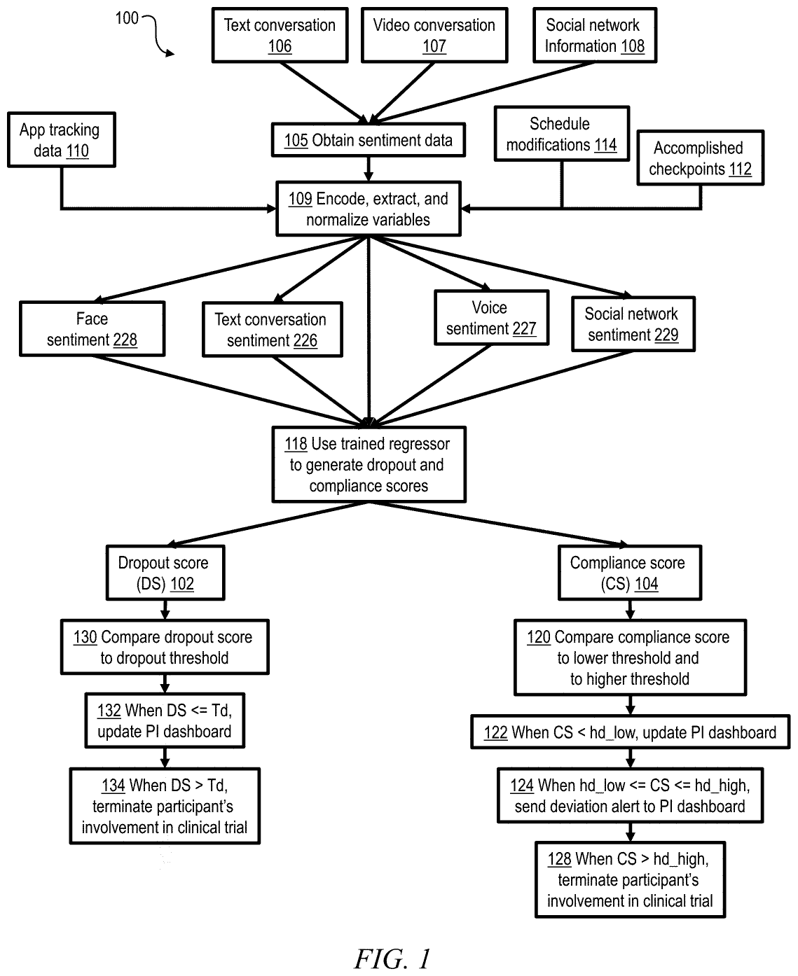

FIG. 1 depicts in a flowchart steps of a method 100 for predicting a participant's dropout score 102 and compliance score 104 for a clinical trial, according to an exemplary embodiment. The method 100 includes, at 105, obtaining participant sentiment data including at least one of: a text conversation 106 between a participant and an investigator in a clinical trial; a video conversation 107 between the participant and the investigator; and the participant's social network information 108. Then at 109, a further step includes encoding, extracting, and normalizing variables that include: text conversation sentiment score 226, voice sentiment score 227, face sentiment score 228, and social networks sentiment score 229 (e.g., anger, fear, joy, sadness, analytical, confident, tentative, excited, subdued) obtained from analysis of the participant sentiment data; clinical trial application usage records (app tracking data) 110; accomplished checkpoints 112; and schedule modifications 114. One non-limiting example of a natural language processing analytics engine, useful for estimating sentiments, is IBM's Watson® Tone Analyzer, which can handle text or speech inputs. (WATSON is a registered trademark of International Business Machines Corporation of Armonk, New York.) At 118, use a trained regressor on the normalized variables to generate the dropout score (DS) 102 and the compliance score (CS) 104. The variables are normalized and the normalization weights are proportional to the risk of dropout or the likelihood of compliance. The input vectors have variables about the user engagement (excited, frustrated, impolite, polite, sad, satisfied, sympathetic, etc.), facial expression analysis (anger, joy, sadness), and proportional information about accomplished checkpoints in the clinical trial. At 120, compare the compliance score 104 to a lower threshold h_{d_low} and to a higher threshold h_{d_high}. When the compliance score is less than h_{d_low}, at 122 update a principal investigator/sponsor dashboard (“PI dashboard”) with a new value of the compliance score 104. When the compliance score is between h_{d_low}, and h_{d_high} inclusive, at 124 send a deviation alert to the PI dashboard with the score, insights, and suggestions of possible interventions. The possible interventions are generated based on past profiles of similar participants and the severity of the compliance scores, the interventions range from schedule a video calls with the PI and the participant, a text conversation or a physical appointment. When the compliance score exceeds h_{d_high}, at 128 send an alert to the PI dashboard and terminate the participant's involvement in the clinical trial. Also, at 130 compare the dropout score 102 to a dropout threshold T_{d}. When the dropout score 102 is less than or equal to T_{d}, then at 132 update the PI dashboard with a new value of the dropout score 102. When the dropout score 102 exceeds T_{d}, then at 134 send an alert to the PI dashboard and terminate the participant's involvement in the clinical trial. Please note that blocks 120 and 130 are decision blocks but are depicted as ordinary operations to save space on the flow chart.

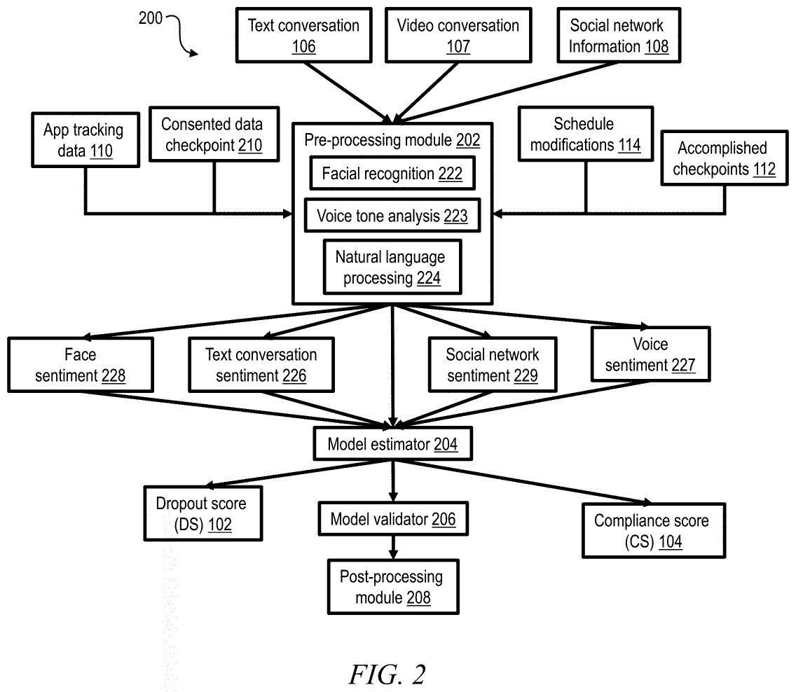

FIG. 2 depicts schematically a system (risk engine) 200 for implementing the method of FIG. 1, according to an exemplary embodiment. The risk engine 200 includes a pre-processing module 202, a model estimator 204, a model validator 206, and a post-processing module 208.

The pre-processing module 202 receives consented data checkpoints 210 (i.e. participant-provided clinical trial monitoring data for which the participant has permitted use/processing of the data by the system through explicit consent), participant-investigator text conversations 106, participant-investigator videos 107, participant social media posts 108 (which may include video posts, which would be processed similarly to the participant-investigator videos 107), clinical trial app tracking data 110, accomplished checkpoint history 112, and schedule modifications 114. In this context, a “clinical trial app” refers to software provided to a clinical trial participant, via the participant's mobile device and/or web browser, in order to enhance the participant's experience of the clinical trial by, e.g., enabling timely communication between the participant and a trial investigator; providing reminders to the participant regarding required actions such as taking medication.

The pre-processing module 202 applies facial recognition 222 and pitch analysis 223 to the participant videos in order to extract sentiment scores 227, 228 from audible and visible aspects of the videos. As a non-limiting example, in one or more embodiments the pre-processing module 202 may make use of sentiment analysis in a video conference—as taught for example by IBM's U.S. Pat. No. 9,648,061—to generate sentiment score 228 by facial expression analysis and sentiment score 227 by voice tone analysis. In one or more embodiments, the facial expression analysis and voice tone analysis software is trained on a corpus of annotated videos showing facial expressions and voice tones similar to the participant's. The pre-processing module 202 also applies natural language processing 224 (e.g., latent Dirichlet analysis or semantic analysis) in order to extract text sentiment score 226 and social network sentiment score 229 from transcripts of the participant's participant-investigator conversations 106, 107 and social media posts 108. As a non-limiting example, in one or more embodiments, the pre-processing module 202 may make use of tones analysis from IBM's Watson® natural language processing software in order to generate sentiment scores 226, 229. The pre-processing module 202 provides the sentiment scores 226, 227, 228, 229, the clinical trial app tracking data 110, the accomplished checkpoint history 112, and the schedule modifications 114 to the model estimator 204.

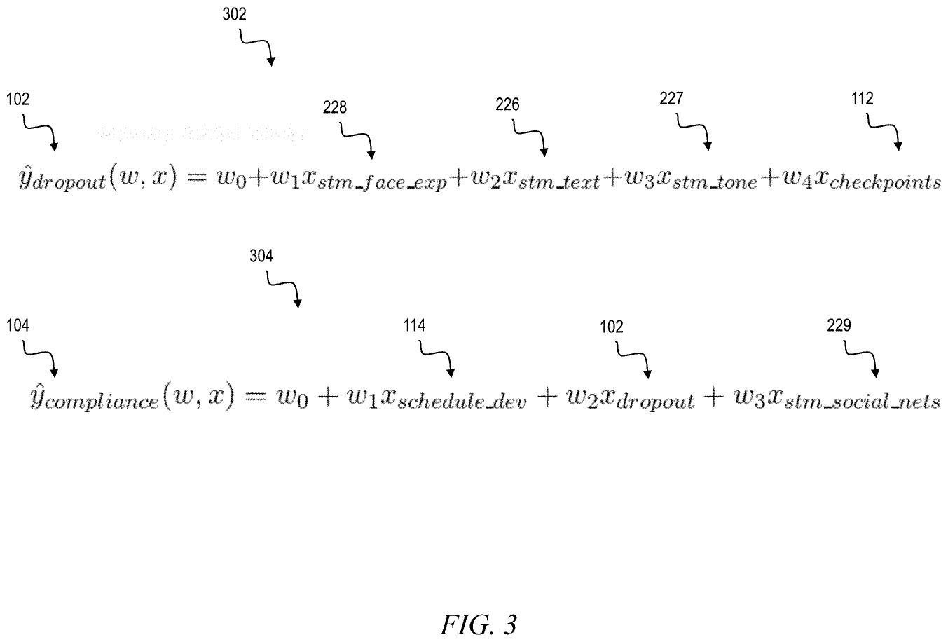

The model estimator 204 is the trained regressor that implements step 118 of the method 100. That is, the model estimator 204 combines the sentiment scores 226, 227, 228, 229, the clinical trial app tracking data 110, the accomplished checkpoint history 112, and the schedule modifications 114 according to formulas 302, 304 that are shown in FIG. 3, in order to generate the dropout score 102 and the compliance score 104. Note that, in one or more embodiments, the dropout score 102 and the compliance score 104 are normalized on a scale 0 . . . 1 with 0 being lowest risk of dropout or other non-compliance and 1 being highest risk.

The model validator 206 evaluates the online trained model on a test data set to assert the ability of the newly trained model estimator to generalize to previously unseen data.

The post-processing module 208 implements steps 120-134 of the method 100. That is, the post-processing module 208 compares the dropout score and the compliance score to thresholds established based on historical data about trial protocol adherence. The post-processing module also is responsible to select what information and scores are presented to the dashboard PI.

FIG. 3 depicts formulas 302, 304 for calculating a participant's dropout score 102 and compliance score 104. Formula 302 is for calculating a dropout score 102 (ŷ_{dropout}) by adjusting a baseline risk of participant dropout w₀ by applying a vector of coefficients w₁ . . . w₄ to an input vector that includes a participant-investigator conversations text sentiment score 226 (x_{stm_text}), a voice tone sentiment score 227 (x_{stm_tone}), a facial expression sentiment score 228 (x_{stm_face_exp}), as well as a checkpoints history score 112 (x_{checkpoints}). Formula 304 is for calculating a compliance score 104 (ŷ_{compliance}) by adjusting a baseline risk of participant non-compliance w₀ by applying a vector of weights w₁ . . . w₃ to an input vector that includes a schedule deviation score 114 (x_{schedule_dev}), the dropout score 102 (ŷ_{dropout}), and a social media text sentiment score 229 (x_{stm_social_nets}).

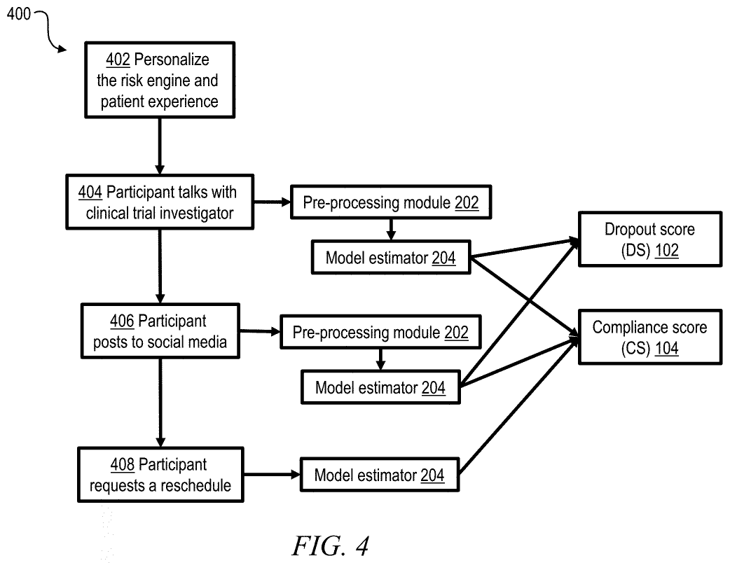

FIG. 4 depicts schematically an example 400 of operation of the method and system of FIGS. 1 and 2. At 402, personalize a participant's patient experience based on the participant's social networks and calendar. The calendar schedule of the participant is used to avoid overlap of personal meetings with clinical trial appointments. The social networks accounts are used to track participant engagement with the clinical trial, as discussed for example in the published patent application “Modeling user attitudes toward a target from social media” (US20150347905A1). This input is useful for the compliance score xₛₜm social nets. At 404, the participant talks with a clinical trial investigator (the participant's “doctor companion”); during the conversation, the pre-processing module 202 captures video and audio and extracts sentiment scores 226, 227 as discussed with reference to FIG. 2. For example, from facial expression a score on 0.83 in the “joy” sentiment is extracted. From audio data an engagement tone is obtained as “excited” with a score of 0.9. These scores are fed to the model estimator 204, generating a decrease on the current dropout score 102. At 406, the participant posts on social media that s/he is very happy to see her/his glucose levels normalized after two months in the CT. Also the participant shares a link on ten facts about diabetes and healthy eating. From these posts the pre-processing module 202 generates a sentiment score 228 for positive engagement 0.86, and the model estimator 204 reduces the compliance score 104 by a corresponding delta value. However, at 408 the participant misses her/his timed dose of medication and requests a two hour reschedule. The deviation is not critical according to the clinical trial protocol, but results in the model estimator 204 slightly increasing the compliance score 104.

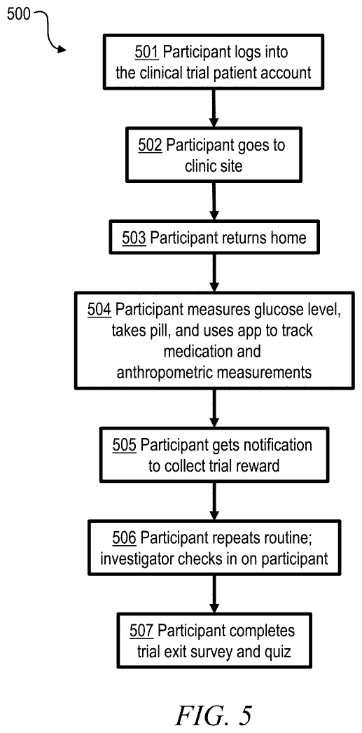

FIG. 5 depicts in a flowchart another example 500 of how an embodiment of the invention works.

In step 501, the participant logs in to the participant's Clinical Trial (CT) Patient account. During this process, the participant agrees to link the participant's FACEBOOK and TWITTER accounts and calendar schedules to the app to personalize the participant's retention scheduler and patient experience.

In step 502, the participant goes to the clinic site where the participant's baseline HbA1c measurement is taken at the lab (using the Digital Health Wallet clinical workflow). The participant's blockchain-verified lab result is recorded on the CT Manager and is also made available on the participant's patient app. The participant can share the participant's data based on the rules specified on the smart contract.

In step 503, the participant returns home to begin the site-less phase of the Clinical Trial. The participant opens the DHW CT Patient App and is able to see the participant's personalized tasks, milestones, checkpoints for Day 1 and can also view the same for Week 1.

In step 504, on “Day One,” the participant wakes up, measures the participant's glucose level, and takes the participant's pill according to the participant's scheduler recommendation. The participant uses the app to confirm that the participant took the medication as instructed. The participant takes the participant's anthropometric measurements (Height, Weight, Waist, BP etc.) as specified in the training material. The participant uses the virtual assistant to take the participant's measurements.

The participant wears a FITBIT or similar activity tracker to track the participant's daily activity. At midday, the participant steps out for lunch. The participant takes a picture of the food the participant plans to eat and the picture is securely saved on the DHW CT Patient App.

In step 505, after one week, the participant gets a notification that the participant can remotely collect the participant's first trial reward (voucher, bitcoin, etc.) based on the smart contract and protocol compliance.

In step 506, this routine is repeated every day for the remainder of the clinical trial. At scheduled times, specified on the Protocol, an investigator uses the DHW Conversations module to call the participant and to check in on the participant's progress. The investigator uses a standardized form to assess the participant's progress.

In step 507, at the end of the clinical trial, the participant completes a trial exit survey and final quiz; afterwards the participant can receive the participant's final reward.

Given the discussion thus far, it will be appreciated that, in general terms, an exemplary method, according to an aspect of the invention, includes at 105 obtaining participant sentiment data including at least one of: a text conversation 106 between a participant and an investigator in a clinical trial; a video conversation 107 between the participant and the investigator; and the participant's social network information 108; at 109 extracting and normalizing a sentiment score from the participant sentiment data; at 118 generating a compliance score for the participant by using a trained regressor on at least the sentiment score; at 120 comparing the compliance score to a lower threshold and to a higher threshold; at 122-128, selecting an action from a decision tree. The decision tree includes: in case the compliance score is less than the lower threshold, updating a principal investigator's dashboard with the compliance score; in case the compliance score is greater than or equal to the lower threshold and less than or equal to the higher threshold, updating the principal investigator's dashboard with a deviation alert and the compliance score; and in case the compliance score exceeds the higher threshold, sending a termination alert to the principal investigator's dashboard and terminating the participant's involvement in the clinical trial. A further step includes facilitating the action.

In one or more embodiments, the trained regressor generates the compliance score and also displays which factors contribute more to the compliance score.

In one or more embodiments, the sentiment score is extracted from the participant sentiment data using natural language processing. Also, the sentiment score can be extracted from the participant sentiment data using at least one of facial recognition and pitch analysis.

In one or more embodiments, the method also includes using the trained regressor on a combination of the sentiment score with at least one of the participant's clinical trial app tracking data, the participant's accomplished checkpoint history, and the participant's clinical trial schedule modifications.

In one or more embodiments, the method also includes generating a dropout score for the participant by using the trained regressor on the sentiment score; comparing the dropout score to a dropout threshold; and in response to the dropout score being less than the dropout threshold, sending an alert to the principal investigator's dashboard with the dropout score. On the other hand, the method also can include, in response to the dropout score exceeding the dropout threshold, terminating the participant's involvement in the clinical trial.

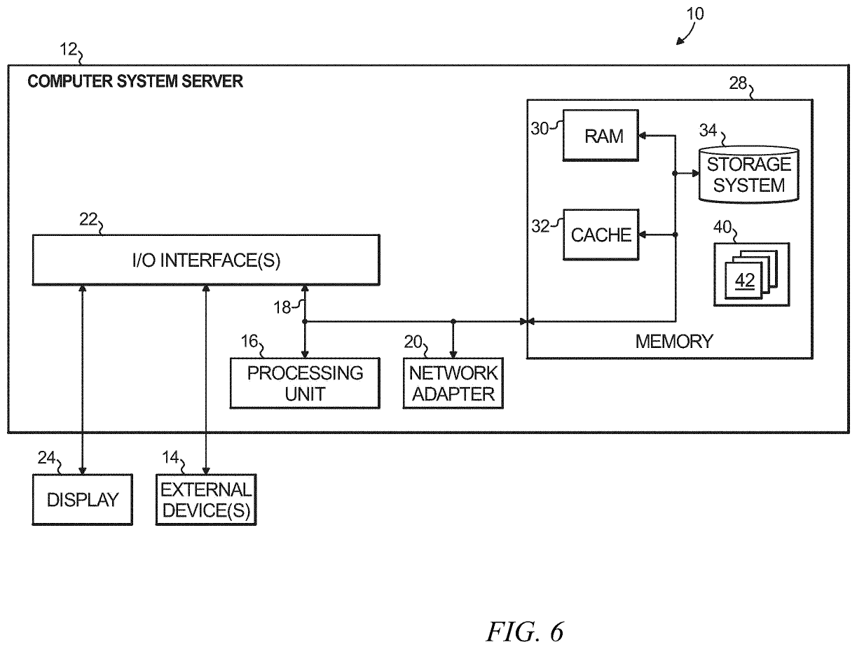

One or more embodiments of the invention, or elements thereof, can be implemented in the form of an apparatus including a memory and at least one processor that is coupled to the memory and operative to perform or otherwise facilitate exemplary method steps, or in the form of a non-transitory computer readable medium embodying computer executable instructions which when executed by a computer cause the computer to perform or to otherwise facilitate exemplary method steps. FIG. 6 depicts a computer system that may be useful in implementing one or more aspects and/or elements of the invention, also representative of a cloud computing node according to an embodiment of the present invention. Referring now to FIG. 6, cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove. Note that both cloud and non-cloud embodiments are contemplated, as well as embodiments with some cloud functionality and some non-cloud functionality. Node 10 is generally representative of a computing device usable in both cloud and non-cloud implementations.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 6, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, and external disk drive arrays, RAID systems, tape drives, and data archival storage systems, etc.

Thus, one or more embodiments can make use of software running on a general purpose computer or workstation. With reference to FIG. 6, such an implementation might employ, for example, a processor 16, a memory 28, and an input/output interface 22 to a display 24 and external device(s) 14 such as a keyboard, a pointing device, or the like. The term “processor” as used herein is intended to include any processing device, such as, for example, one that includes a CPU (central processing unit) and/or other forms of processing circuitry. Further, the term “processor” may refer to more than one individual processor. The term “memory” is intended to include memory associated with a processor or CPU, such as, for example, RAM (random access memory) 30, ROM (read only memory), a fixed memory device (for example, hard drive 34), a removable memory device (for example, diskette), a flash memory and the like. In addition, the phrase “input/output interface” as used herein, is intended to contemplate an interface to, for example, one or more mechanisms for inputting data to the processing unit (for example, mouse), and one or more mechanisms for providing results associated with the processing unit (for example, printer). The processor 16, memory 28, and input/output interface 22 can be interconnected, for example, via bus 18 as part of a data processing unit 12. Suitable interconnections, for example via bus 18, can also be provided to a network interface 20, such as a network card, which can be provided to interface with a computer network, and to a media interface, such as a diskette or CD-ROM drive, which can be provided to interface with suitable media.

Accordingly, computer software including instructions or code for performing the methodologies of the invention, as described herein, may be stored in one or more of the associated memory devices (for example, ROM, fixed or removable memory) and, when ready to be utilized, loaded in part or in whole (for example, into RAM) and implemented by a CPU. Such software could include, but is not limited to, firmware, resident software, microcode, and the like.

A data processing system suitable for storing and/or executing program code will include at least one processor 16 coupled directly or indirectly to memory elements 28 through a system bus 18. The memory elements can include local memory employed during actual implementation of the program code, bulk storage, and cache memories 32 which provide temporary storage of at least some program code in order to reduce the number of times code must be retrieved from bulk storage during implementation.

Input/output or I/O devices (including but not limited to keyboards, displays, pointing devices, and the like) can be coupled to the system either directly or through intervening I/O controllers.

Network adapters 20 may also be coupled to the system to enable the data processing system to become coupled to other data processing systems or remote printers or storage devices through intervening private or public networks. Modems, cable modem and Ethernet cards are just a few of the currently available types of network adapters.

As used herein, including the claims, a “server” includes a physical data processing system (for example, system 12 as shown in FIG. 6) running a server program. It will be understood that such a physical server may or may not include a display and keyboard.

One or more embodiments can be at least partially implemented in the context of a cloud or virtual machine environment, although this is exemplary and non-limiting.

It should be noted that any of the methods described herein can include an additional step of providing a system comprising distinct software modules embodied on a computer readable storage medium; the modules can include, for example, any or all of the appropriate elements depicted in the block diagrams and/or described herein; by way of example and not limitation, any one, some or all of the modules/blocks and or sub-modules/sub-blocks described. The method steps can then be carried out using the distinct software modules and/or sub-modules of the system, as described above, executing on one or more hardware processors such as 16. Further, a computer program product can include a computer-readable storage medium with code adapted to be implemented to carry out one or more method steps described herein, including the provision of the system with the distinct software modules.

One example of user interface that could be employed in some cases is hypertext markup language (HTML) code served out by a server or the like, to a browser of a computing device of a user. The HTML is parsed by the browser on the user's computing device to create a graphical user interface (GUI).

Exemplary System and Article of Manufacture Details

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
obtaining participant sentiment data including at least one of: a text conversation between a participant and an investigator in a clinical trial; a video conversation between the participant and the investigator; and the participant's social network information;
extracting and normalizing a sentiment score from the participant sentiment data;
generating a compliance score for the participant by using a trained regressor on at least the sentiment score;
comparing the compliance score to a lower threshold and to a higher threshold;
selecting an action from a decision tree, the decision tree comprising:
in case the compliance score is less than the lower threshold, updating a principal investigator's dashboard with the compliance score;
in case the compliance score is greater than or equal to the lower threshold and less than or equal to the higher threshold, updating the principal investigator's dashboard with a deviation alert and the compliance score; and
in case the compliance score exceeds the higher threshold, sending a termination alert to the principal investigator's dashboard and terminating the participant's involvement in the clinical trial; and
facilitating the action.

2. The method of claim 1 wherein the trained regressor generates the compliance score and also displays which factors contribute more to the compliance score.

3. The method of claim 1 wherein the sentiment score is extracted from the participant sentiment data using natural language processing.

4. The method of claim 1 wherein the sentiment score is extracted from the participant sentiment data using at least one of facial recognition and pitch analysis.

5. The method of claim 1 further comprising using the trained regressor on a combination of the sentiment score with at least one of the participant's clinical trial app tracking data, the participant's accomplished checkpoint history, and the participant's clinical trial schedule modifications.

6. The method of claim 1 further comprising:
generating a dropout score for the participant by using the trained regressor on the sentiment score;
comparing the dropout score to a dropout threshold; and
in response to the dropout score being less than the dropout threshold, sending an alert to the principal investigator's dashboard with the dropout score.

7. The method of claim 1 further comprising:
generating a dropout score for the participant by using the trained regressor on the sentiment score;
comparing the dropout score to a dropout threshold; and
in response to the dropout score exceeding the dropout threshold, terminating the participant's involvement in the clinical trial.

8. A non-transitory computer readable medium embodying computer executable instructions which when executed by a computer cause the computer to facilitate a method of:
obtaining participant sentiment data including at least one of: a text conversation between a participant and an investigator in a clinical trial; a video conversation between the participant and the investigator; and the participant's social network information;
extracting and normalizing a sentiment score from the participant sentiment data;
generating a compliance score for the participant by using a trained regressor on at least the sentiment score;
comparing the compliance score to a lower threshold and to a higher threshold;
selecting an action from a decision tree, the decision tree comprising:
in case the compliance score is less than the lower threshold, updating a principal investigator's dashboard with the compliance score;
in case the compliance score is greater than or equal to the lower threshold and less than or equal to the higher threshold, updating the principal investigator's dashboard with a deviation alert and the compliance score; and
in case the compliance score exceeds the higher threshold, sending a termination alert to the principal investigator's dashboard and terminating the participant's involvement in the clinical trial; and
facilitating the action.

9. The non-transitory computer readable medium of claim 8 wherein the trained regressor generates the compliance score and also displays which factors contribute more to the compliance score.

10. The non-transitory computer readable medium of claim 8 wherein the sentiment score is extracted from the participant sentiment data using natural language processing.

11. The non-transitory computer readable medium of claim 8 wherein the sentiment score is extracted from the participant sentiment data using at least one of facial recognition and pitch analysis.

12. The non-transitory computer readable medium of claim 8, wherein the method further comprises using the trained regressor on a combination of the sentiment score with at least one of the participant's clinical trial app tracking data, the participant's accomplished checkpoint history, and the participant's clinical trial schedule modifications.

13. The non-transitory computer readable medium of claim 8 wherein the method further comprises:
generating a dropout score for the participant by using the trained regressor on the sentiment score;
comparing the dropout score to a dropout threshold; and
in response to the dropout score being less than the dropout threshold, sending an alert to the principal investigator's dashboard with the dropout score.

14. An apparatus comprising:
a memory embodying computer executable instructions; and
at least one processor, coupled to the memory, and operative by the computer executable instructions to facilitate a method of:
obtaining participant sentiment data including at least one of: a text conversation between a participant and an investigator in a clinical trial; a video conversation between the participant and the investigator; and the participant's social network information;
extracting and normalizing a sentiment score from the participant sentiment data;
generating a compliance score for the participant by using a trained regressor on at least the sentiment score;
comparing the compliance score to a lower threshold and to a higher threshold;
selecting an action from a decision tree, the decision tree comprising:
in case the compliance score is less than the lower threshold, updating a principal investigator's dashboard with the compliance score;
in case the compliance score is greater than or equal to the lower threshold and less than or equal to the higher threshold, updating the principal investigator's dashboard with a deviation alert and the compliance score; and
in case the compliance score exceeds the higher threshold, sending a termination alert to the principal investigator's dashboard and terminating the participant's involvement in the clinical trial; and
facilitating the action.

15. The apparatus of claim 14 wherein the trained regressor generates the compliance score and also displays which factors contribute more to the compliance score.

16. The apparatus of claim 14 wherein the sentiment score is extracted from the participant sentiment data using natural language processing.

17. The apparatus of claim 14 wherein the sentiment score is extracted from the participant sentiment data using at least one of facial recognition and pitch analysis.

18. The apparatus of claim 14 wherein the method further comprises using the trained regressor on a combination of the sentiment score with at least one of the participant's clinical trial app tracking data, the participant's accomplished checkpoint history, and the participant's clinical trial schedule modifications.

19. The apparatus of claim 14 wherein the method further comprises:
generating a dropout score for the participant by using the trained regressor on the sentiment score;
comparing the dropout score to a dropout threshold; and
in response to the dropout score being less than the dropout threshold, sending an alert to the principal investigator's dashboard with the dropout score.

20. The apparatus of claim 14 wherein the method further comprises:
generating a dropout score for the participant by using the trained regressor on the sentiment score;
comparing the dropout score to a dropout threshold; and
in response to the dropout score exceeding the dropout threshold, terminating the participant's involvement in the clinical trial.
